(19) United States
(12) Patent Application Publication
(10) Pub. No.: US 2025/0129039 A1
(43) Pub. Date: Apr. 24, 2025

(54) NOVEL PROCESS FOR THE PREPARATION OF BILASTINE AND INTERMEDIATES THEREOF

(71) Applicant: BIOPHORE INDIA PHARMACEUTICALS PVT. LTD., Hyderabad (IN)

(72) Inventors: Manik Reddy PULLAGURLA, Hyderabad (IN); Bhaskar Reddy PITTA, Hyderabad (IN); Jagadeesh Babu RANGISETTY, Hyderabad (IN)

(21) Appl. No.: 18/684,903

(22) PCT Filed: Aug. 19, 2022
(86) PCT No.: PCT/IB2022/057787

§ 371 Date: Feb. 20, 2024

(30) Foreign Application Priority Data
Aug. 19, 2021 (IN) 202141037570

(51) Int. Cl. C07D 401/04; C07C 69/34

(52) U.S. Cl. CPC C07D 401/04; C07C 69/34; C07C 2601/16

(57) ABSTRACT

A novel process for the preparation of 2-[4-[2-[4-[1-(2-ethoxyethyl) benzimidazol-2-yl] piperidin-1-yl] ethyl]phenyl]-2-methyl propionic acid (I) is provided. A process for the purification of Bilastine (I), having purity greater than 99.0% by High performance liquid chromatography (HPLC) is also provided.

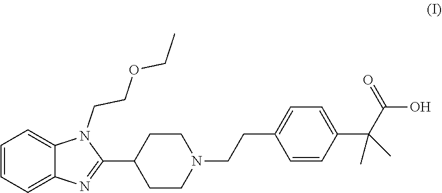 [SMILES: CCOCCn1c(C2CCN(CCc3ccc(C(C)(C)C(=O)O)cc3)CC2)nc2ccccc21]

### CROSS-REFERENCE TO RELATED APPLICATIONS

This application claims priority to PCT Application No. PCT/IB2022/057787, having a filing date of Aug. 19, 2022, which is based on IN 202141037570, having a filing date of Aug. 19, 2021, the entire contents both of which are hereby incorporated by reference.

### FIELD OF TECHNOLOGY

The following relates to a novel process for the preparation of 2-[4-[2-[4-[1-(2-ethoxyethyl) benzimidazol-2-yl]piperidin-1-yl]ethyl]phenyl]-2-methyl propionic acid (I), which is hereinafter referred to as Bilastine (I) through a novel intermediate compound of formula (VII).

The following also relates to a process for the purification of Bilastine (I).

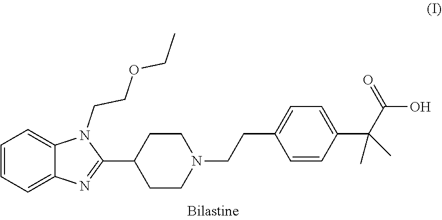 [SMILES: CCOCCn1c(C2CCN(CCc3ccc(C(C)(C)C(=O)O)cc3)CC2)nc2ccccc21]

### BACKGROUND

Bilastine (I), a novel second-generation H1-antihistamine, is approved for the symptomatic treatment of allergic rhino conjunctivitis and urticaria in adults and children over 12 years of age.

U.S. Pat. No. 5,877,187 patent discloses the preparation of Bilastine (I) as depicted in below scheme 1.

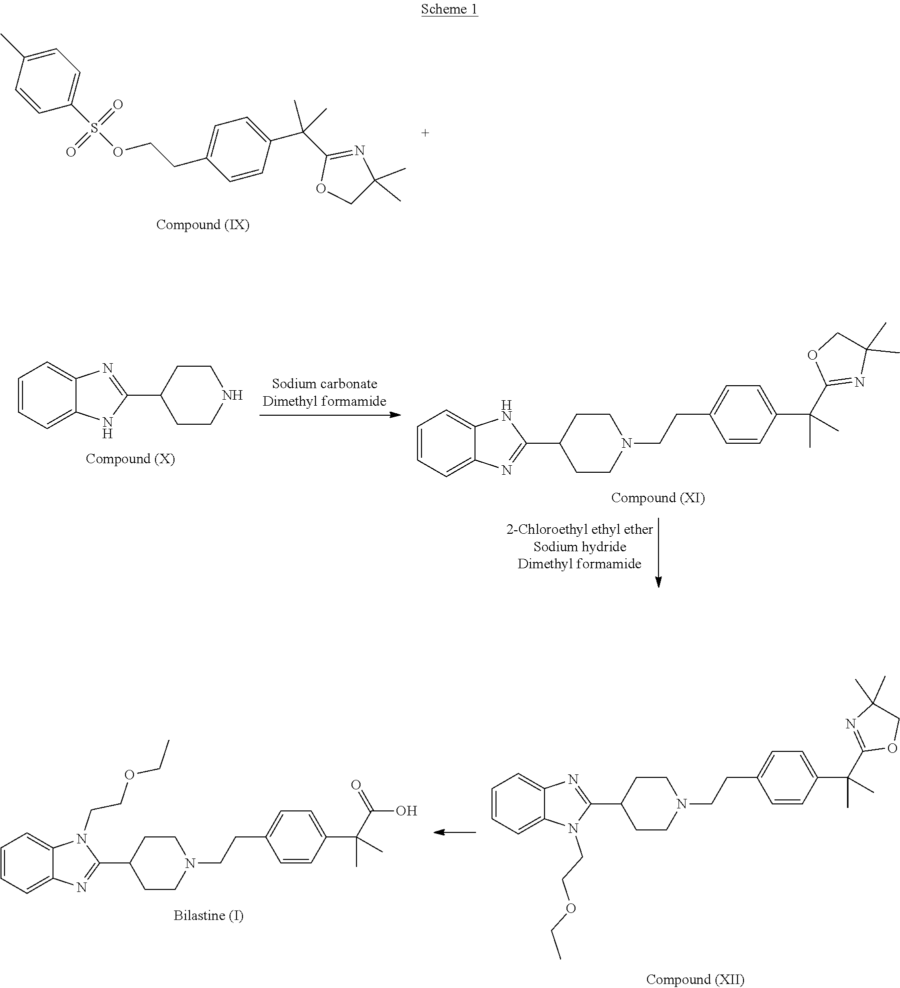

Various process for the preparation of Bilastine (I) and its intermediates were disclosed in U.S. Pat. Nos. 5,877,187, 8,367,704, PCT publications WO2014188453, WO2014026657.

The above synthesis process depicted in U.S. Pat. No. 5,877,187 patent suffers from poor yield due to formation of several by products and involves the use of metal hydrides which are not suitable for the commercial scale. Hence, the present inventors developed an economically viable process for the preparation of Bilastine (I) and intermediate with high yield and purity.

### SUMMARY

An aspect relates to a novel process for the preparation of Bilastine (I), having purity greater than 99.0% by High performance liquid chromatography (HPLC).

In another aspect embodiments of the present invention provide a novel process for the preparation of Bilastine (I) using novel compound of formula (VII).

In another aspect embodiments of the present invention provide a novel intermediate compound of formula (VII).

In yet another aspect embodiments of the present invention provide a process for the purification of Bilastine (I).

Embodiments of the present invention relate to a novel process for the preparation of Bilastine (I), is having purity greater than 99.0% by High performance liquid chromatography (HPLC).

In another aspect embodiments of the present invention provide a novel process for the preparation of Bilastine (I) as shown in scheme 2, comprising:
a) acetylation of 2-phenylethanol (V) in presence of suitable acetylating reagent to form compound (VI),
b) conversion of compound (VI) to compound (VII) in presence of suitable oxidising reagent and a metal halide,
c) hydrolysis of compound (VII) in presence of suitable hydrolysing reagent to form compound (VIII),
d) conversion of compound (VIII) in presence of suitable reagent to form compound (II),
e) coupling of compounds (II) with 1-(2-ethoxyethyl)-2-(piperidin-4-yl)-1H-benzo[d]imidazole (III) in presence of suitable base to form compound (IV), and
f) hydrolysis of compound (IV) to obtain Bilastine (I) in presence of suitable base.

In another aspect embodiments of the present invention provide novel intermediate compound of formula (VII).

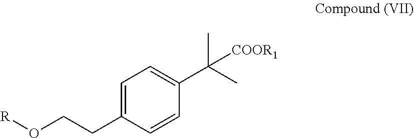

where R is defined as COCH(CH₃)₂, or COCH₃, R₁ is C₁-C₄ alkyl.

In another aspect embodiments of the present invention provide novel intermediate compounds of formula (VII), which include

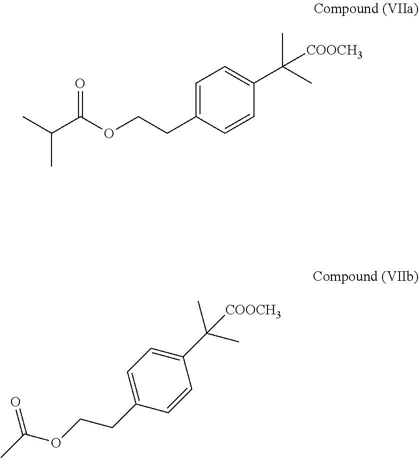 [SMILES: COC(=O)C(C)(C)c1ccc(CCOC(=O)C(C)C)cc1 ; COC(=O)C(C)(C)c1ccc(CCOC(C)=O)cc1]

In another aspect embodiments of the present invention provide novel intermediate compound of formula (VII), used in the preparation of Bilastine (I).

In another aspect embodiments of the present invention provide a process for the purification of Bilastine (I), comprising:
i. providing Bilastine (I), in a suitable solvent.
ii. heating the reaction mixture to a suitable temperature.
iii. cooling the reaction mass; and
iv. isolating pure Bilastine (I).

### DETAILED DESCRIPTION

Embodiments of the present invention relate to a novel process for the preparation of Bilastine (I), is having purity greater than 99.0% by High performance liquid chromatography (HPLC).

In another aspect embodiments of the present invention provide a novel process for the preparation of Bilastine (I) as illustrated in scheme 2.

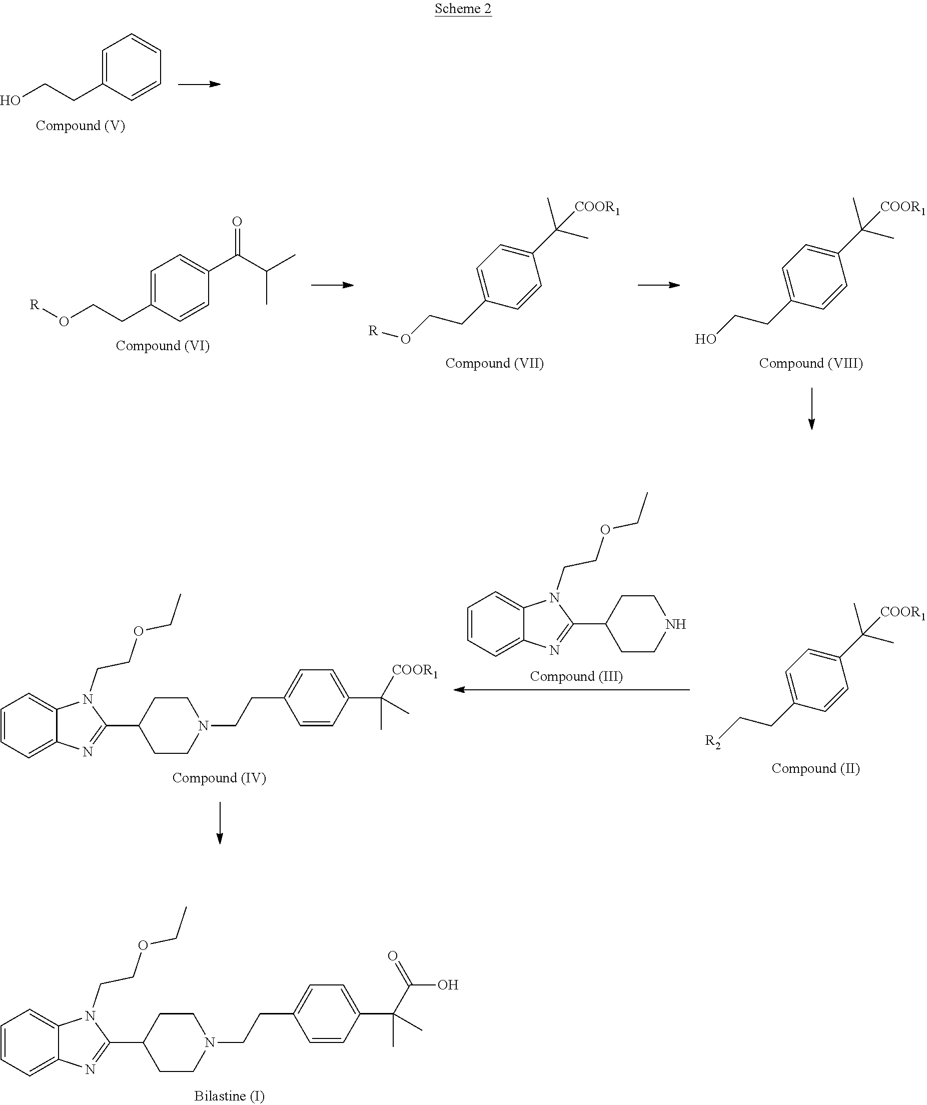

where R is defined as COCH(CH₃)₂ or COCH₃, R₁ is C₁-C₄ alkyl, R₂ is halo or SO₃CH₃

Step a) involves acetylation of 2-phenylethanol (V) in presence of suitable acetylating agent to form compound (VI). The reaction may be carried out at a temperature of 0-30° C., for example 0-5° C. Suitable acetylating agent used in the present reaction is selected from the isobutyryl chloride, isobutyl bromide, acetyl chloride, acetyl bromide, acetic anhydride for example isobutyryl chloride, acetic anhydride used in embodiments of the present invention.

Step b) proceeds conversion of compound (VI) in presence of suitable oxidative reagent and a metal halide to form compound (VII). The suitable oxidative reagent used in embodiments of the present invention can be selected from the group comprising of Iodine (I₂)/tert-butyl hydroperoxide (TBHP), phenyliodonium diacetate (PhI(OAc)₂)/(2,2,6,6-tetramethylpiperidin-1-yl) oxidanyl (TEMPO), Bromine (Br₂)/trimethyl orthoformate (TMOF)/zinc (II) bromide (ZnBr₂), (phenyliodonium diacetate (PhI(OAc)₂) in trimethyl orthoformate, iodine (I₂)/trimethyl orthoformate (TMOF)/sulfuric acid, iodine chloride/trimethyl orthoformate (TMOF) for example iodine chloride/trimethyl orthoformate (TMOF).

The “suitable metal halide” used in step b) can be selected from the group but not limited to iodine chloride, sodium chloride, sodium bromide, sodium iodide, potassium chloride, potassium bromide, potassium iodide, for example iodine chloride.

Step c) involves hydrolysis of compound (VII) in presence of suitable base and solvent to form compound (VIII); suitable base used in step c) may be selected from sodium hydroxide, potassium hydroxide, lithium hydroxide, for example sodium hydroxide.

Suitable solvent used in step c) selected from water, methanol, ethanol, propanol, isopropanol, n-butanol. For example, methanol was used in embodiments of the present invention.

Step d) involves conversion of compound (VIII) in presence of suitable reagent to form compound (II); the suitable reagent can be chlorinating agent or sulfinyl reagent. Wherein suitable chlorinating agent used is selected from the group phosphoryl chloride, thionyl chloride, oxalyl chloride, methane sulfonyl chloride, trichloromethane sulfonyl chloride, for example, phosphoryl chloride, and thionyl chloride were used in embodiments of the present invention, Sulfinyl reagent selected from methane sulfonyl chloride, p-toluenesulfonylchloride, mesyl chloride, p-toluenesulfonyl bromide, methane sulfonyl bromide, for example methane sulfonyl chloride used in present reaction.

Step e) involves coupling of compounds (II) with 1-(2-ethoxyethyl)-2-(piperidin-4-yl)-1H-benzo[d]imidazole (III) in presence of suitable base to form compound (IV). The suitable base used in step e) is selected from the group consisting of hydroxides of alkali and alkaline metals such as lithium hydroxide, sodium hydroxide, potassium hydroxide and the like; carbonates of alkali and alkaline metals such as sodium carbonate, potassium carbonate and the like; and bicarbonates of alkali and alkaline metals such as sodium bicarbonate, potassium bicarbonate and the like.

Step f) involves hydrolysis of compound (IV) to form Bilastine (I) in presence of suitable base. The suitable base used in step f) is selected from the group consisting of hydroxides of alkali and alkaline metals such as lithium hydroxide, sodium hydroxide, potassium hydroxide and the like; carbonates of alkali and alkaline metals such as sodium carbonate, potassium carbonate and the like; and bicarbonates of alkali and alkaline metals such as sodium bicarbonate, potassium bicarbonate and the like.

In an embodiment of the present invention there is provided a novel compound formula (VII).

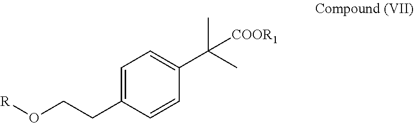

where R is defined as COCH(CH₃)₂, or COCH₃, R₁ is C₁-C₄ alkyl.

In an embodiment of the present invention there is provided examples of desired novel compounds of formula (VII) including

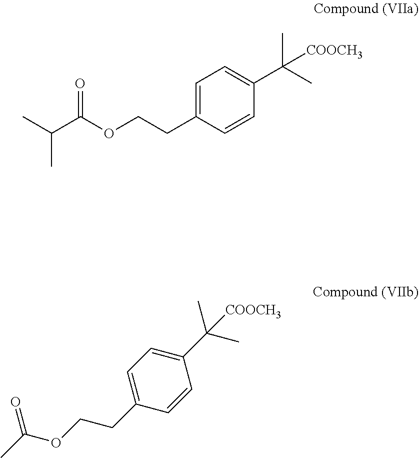 [SMILES: COC(=O)C(C)(C)c1ccc(CCOC(=O)C(C)C)cc1 ; COC(=O)C(C)(C)c1ccc(CCOC(C)=O)cc1]

In an embodiment of the present invention there is provided the novel compound of formula (VII) useful in the preparation of Bilastine (I).

In an embodiment of the present invention there is provided a process for the purification of Bilastine (I), comprising:
i. providing Bilastine (I), in suitable solvent,
ii. heating the reaction mixture to a suitable temperature,
iii. cooling the reaction mass, and
iv. isolating pure Bilastine (I).

The suitable solvents used for the purification of Bilastine (I) may be selected from a group comprising of protic solvents. Protic solvents may comprise of water, methanol, ethanol, propanol, isopropanol, n-butanol, or mixtures thereof. For example, water, methanol, isopropyl alcohol was used in embodiments of the present invention.

In an embodiment, Bilastine obtained according to embodiments of the present invention is having moisture content not more than 4%, for example not more than 2%.

In an embodiment, Bilastine obtained according to embodiments of the present invention is having following elemental analysis, which is shown below:

In an embodiment, Bilastine obtained after purification is having purity greater than 99% by HPLC and total impurities less than 1.0% (w/w), for example less than 0.5% and as a further example less than 0.15%. Further any unknown impurity is controlled less than 0.10%.

The process described in embodiments of the present invention is demonstrated in examples illustrated below. These examples are provided as illustration only and therefore should not be construed as limitation of the scope of embodiments of the invention.

### EXAMPLES

### Example 1: Preparation of Compound (VI) (where R═COCH(CH₃)₂)

160 g Isobutyryl chloride was added to 500 ml of dichloromethane and cooled the reaction mixture to 0-5° C. 150 g of aluminium chloride was slowly added to the reaction mixture at 0-5° C. Then 100 g of 2-phenylethanol (V) was added to the reaction mixture. The reaction mixture was stirred for 4 hr. To the solid hydrochloric acid was added. The solid obtained was dried at 25-40° C. to get 4-isobutyrylphenethyl isobutyrate (VI). Yield: 150 g, Purity: 99.0%

### Example 2: Preparation of Compound (VI) (where R═COCH₃)

400 g of 2-phenylethanol (V) was added to 1000 ml acetic anhydride and then 40 g of dimethyl amino pyridine was added to the reaction mixture at 25-30° C. and stirred for 10 min. The reaction mixture was heated to below 90-95° C. After completion of the reaction, the reaction mass was cooled to 25-30° C. To the reaction mass saturated sodium bicarbonate solution and 800 ml of methyl tertiary butyl ether were added and stirred for 10 min. The organic layer separated, washed with 800 ml of saturated sodium bicarbonate, 800 ml of water. The organic layer was distilled off under vacuum to obtain pure phenethyl acetate.

In another reaction flask 162 g of Isobutyryl chloride, 159 g of aluminium trichloride, and 1 liter dichloromethane were added at-5 to 0° C. The above obtained 100 g phenethyl acetate was added to the reaction mixture at-5 to 0° C. The reaction mixture was stirred for 4 hr. After completion of the reaction 1M hydrochloric acid was added at-5-0° C. The aqueous layer washed with 200 ml of dichloromethane. The organic layer washed with 500 ml of saturated sodium hydroxide and saturated 800 ml of sodium chloride at 20-30° C. The organic layer was distilled off at below 40-45° C. To obtain 4-isobutyrylphenethyl acetate (VI). Yield: 143 g; Purity: 99.0%.

### Example 3: Preparation of Compound (VII a) (where R═COCH(CH₃)₂, R₁═CH₃)

10 g of isobutyrylphenethyl isobutyrate (VI). was charged to 40 ml of trimethyl orthoformate. 7 ml of Iodine chloride was added to the reaction mixture at 20-35° C. The reaction mass was maintained for 1 hr at 20-35° C. After completion of the reaction 200 ml of saturated sodium bicarbonate solution was added and stirred for 10 min. The organic layer washed with 200 ml of sodium thiosulfate solution. The organic layer was distilled off at below 45° C. to get pure methyl 2-(4-(2-(isobutyryloxy) ethyl)phenyl)-2-methylpropanoate (VIIa). Yield: 270 g, Purity: 99.0%.

### Example 4: Preparation of Compound (VII b) (R═COCH₃, R₁=CO₂CH₃)

150 g of 4-isobutyrylphenethyl acetate (VI) was charged to 600 ml of trimethyl orthoformate. 105 ml of Iodine chloride was added to the reaction mixture at 20-35° C. The reaction mass was maintained for 1 hr at 20-35° C. After completion of the reaction 1500 ml of saturated sodium sulphate solution was added and stirred for 10 min. The organic layer washed with 900 ml of sodium bicarbonate solution. The reaction mass washed with 500 ml of sodium chloride solution. The organic layer was distilled off at below 45° C. to get pure methyl 2-(4-(2-acetoxyethyl)phenyl)-2-methylpropanoate (VII). Yield: 150 g; Purity: 99.0%.

### Example 5: Preparation of Compound (VIII) (where R₁═CO₂CH₃)

100 g of compound (VII) was charged to 500 ml of methanol and cooled to 10-15° C. Then 2 g of sodium methoxide was added at 10-15° C. The reaction was maintained for 1 hr at 25-30° C. After completion of the reaction 10 ml of water was added. Methanol was distilled at below 40-45° C. Then 100 ml of water was added to the reaction mass and stirred. The aqueous layer extracted with 500 ml of ethyl acetate and organic layer was washed with 500 ml saturated sodium carbonate solution, 500 ml of sodium chloride solution. The organic layer was distilled off at below 45° C. to get pure methyl 2-(4-(2-hydroxyethyl)phenyl)-2-methylpropanoate (VIII). Yield: 5 g; Purity: 99.0%

### Example 6: Preparation of Compound (II) (where R₁═CO₂CH₃, R₂═Cl)

15 g of methyl 2-(4-(2-hydroxyethyl)phenyl)-2-methylpropanoate (VIII) was charged to 75 ml of toluene and 1.5 ml of dimethyl formamide at 25-30° C. To the reaction mixture 12 g of thionyl chloride was added at 25-30° C. The reaction temperature was raised to below 80-85° C. After completion of reaction, the reaction mixture was cooled to 25-30° C. The reaction mixture was quenched with 150 ml of ice-cold water and stirred for 10 min. The aqueous layer extracted with 350 ml of toluene and organic layer washed with 450 ml of saturated sodium bicarbonate solution. The organic layer was distilled off at 50-55° C. to get 2-(4-(2-chloroethyl)phenyl)-2-methylpropanoate (II). Yield: 14 g; Purity: 99.0%.

### Example 7: Preparation of Compound (II) (where R₁═CO₂CH₃, R₂═SO₃CH₃)

15 g of methyl 2-(4-(2-hydroxyethyl)phenyl)-2-methylpropanoate (VIII) was charged to 75 ml of dichloromethane and cooled to 0-5° C. 11.5 g of methane sulfonyl chloride was added to the reaction mixture at 0-5° C. After completion of the reaction 150 ml of water was added and stirred for 10 min. The organic layer washed with 900 ml of dichloromethane. Then washed with 100 ml of water. The organic layer was distilled off at below 45° C. to get pure methyl 2-methyl-2-(4-(2-(methyl sulfonyl oxy) ethyl)phenyl) propanoate) (II). Yield: 19 g; Purity: 99.0%

### Example 8: Preparation of Compound (IV) (where R₁═CO₂CH₃,)

11 g of 1-(2-ethoxyethyl)-2-(piperidin-4-yl)-1H-benzo[d]imidazole (III) was charged to 30 ml of dimethyl formamide and 17 g of potassium carbonate and then heated to 80-85° C. 10 g of compound (II) was dissolved in 20 ml of dimethyl formamide in another reaction flask and is added to the first reaction mixture at 80-85° C. Then the reaction mixture temperature was raised to 135 to 140° C. After completion of the reaction, reaction mixture was cooled to 20-25° C. To the reaction mixture 500 ml of water and 50 ml of methyl tertiary butyl ether were charged. The reaction mixture pH was adjusted to 1 to 2 using 6N hydrochloric acid and then aqueous and organic layers were separated. The aqueous layer taken in another reaction flask and pH adjusted to 8 to 9 using saturated sodium carbonate solution and then extracted with 200 ml of ethyl acetate. The organic layer washed with 100 ml water. The organic layer was distilled off at below 55° C. to get methyl 2-(4-(2-(4-(1-(2-ethoxyethyl)-1H-benzo[d]imidazol-2-yl) piperidin-1-yl) ethyl)phenyl)-2-methylpropanoate (IV). Yield: 15 g; Purity: 99.0%.

### Example 9: Preparation of 2-(4-(2-(4-(1-(2-ethoxyethyl)-1H-benzo[d]imidazol-2-yl) piperidin-1-yl) ethyl)phenyl)-2-methylpropanoic acid (I)

15 g of compound (IV) was charged to 45 ml of ethanol at 25-30° C. 3.8 g of sodium hydroxide dissolved in 45 ml of water was added to the reaction mixture. The reaction mixture was maintained at 35-40° C. After completion of the reaction 45 ml of ethanol was added. The pH of the aqueous layer was adjusted to 6.5 to 7.5 using hydrochloric acid solution and then 100 ml of ethyl acetate was charged and stirred for 1 hr at 25-30° C. The solid was filtered and washed with 30 ml of ethyl acetate and dried at below 50° C. to obtain 2-(4-(2-(4-(1-(2-ethoxyethyl)-1H-benzo[d]imidazol-2-yl) piperidin-1-yl) ethyl)phenyl)-2-methylpropanoic acid (I). Yield: 8.2 g.

### Example 10: Purification of Bilastine (I)

10 g of Bilastine (I) was added to 50 ml of methanol and heated to 50-60° C., stirred for 10 min. To the reaction mixture 50 ml of acetone was added and stirred at 50-60° C. for 15 minutes. The reaction mass was cooled to 20-25° C. and stirred for 30 minutes. After completion of the reaction, the reaction mass was filtered and washed with 20 ml of methanol. To the filtrate, 30 ml of methanol was added and heated to 50-60° C. and stirred for 30 minutes. 1.0 g of activated carbon was added to the reaction mass and then stirred for 10 minutes. Then the reaction mass was filtered and washed with 10 ml of hot methanol. The filtrate was cooled to 20-22° C. and stirred for 40 minutes at 20-22° C. The solid was filtered and washed with 10 ml of cold methanol and dried at below 60-70° C. to obtain pure Bilastine (I). Yield: 7.0 g; Purity: 99.9%.

### Example 11: Purification of Bilastine (I)

A mixture of 10 g of Bilastine (I) and 75 ml of isopropyl alcohol was heated to 80-85° C. and stirred for 30 min at 80-85° C. The reaction mixture was cooled to 5-10° C. and stirred for 30 min. The obtained solid was filtered, washed with 25 mL isopropyl alcohol, and dried the material to get pure Bilastine (I). Yield: 7.0 g; Purity: 99.89%.

Although the invention has been illustrated and described in greater detail with reference to the preferred exemplary embodiments, the invention is not limited to the examples disclosed, and further variations can be inferred by a person skilled in the art, without departing from the scope of protection of the invention.

For the sake of clarity, it is to be understood that the use of “a” or “an” throughout this application does not exclude a plurality, and “comprising” does not exclude other steps or elements.

1. A process for the preparation of Bilastine (I), comprising:
i. acetylation of 2-phenylethanol (V) in presence of suitable acetylating reagent and aluminium chloride to form compound (VI); 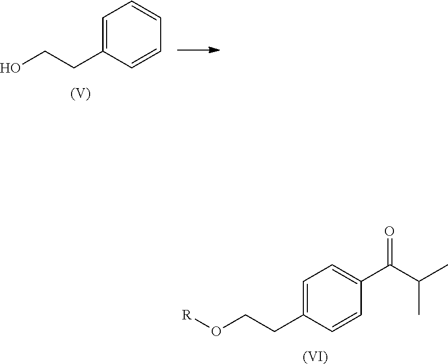
wherein, R is defined as COCH(CH₃)₂ or COCH₃,
ii. conversion of compound (VI) in presence of a suitable oxidative reagent and a metal halide to form compound (VII); 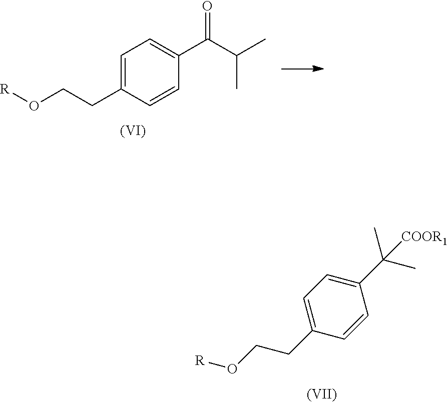
wherein, R is defined as COCH(CH₃)₂ or COCH₃; R₁ is C₁-C₄ alkyl.
iii. hydrolysis of compound (VII) in the presence of a suitable base to form compound (VIII); 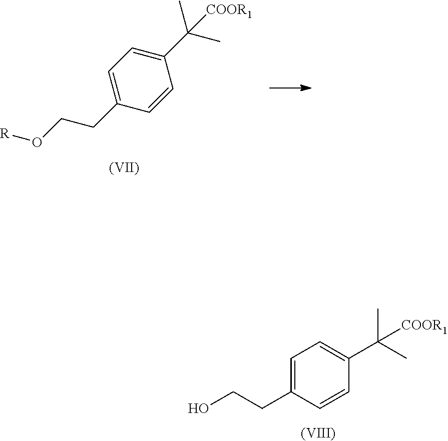
wherein, R is defined as COCH(CH₃)₂ or COCH₃; R₁ is C₁-C₄ alkyl.
iv. conversion of compound (VIII) with a suitable reagent to form compound (II); 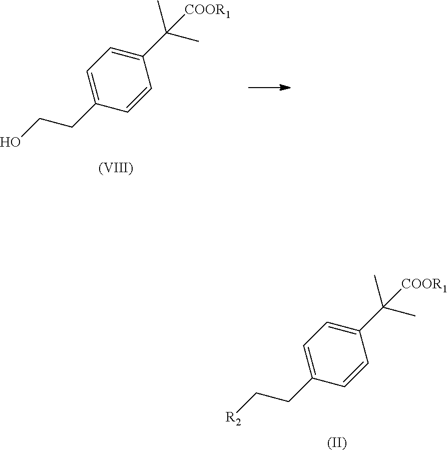
wherein, R₁ is C₁-C₄ alkyl; R₂ is halo or SO₃CH₃
v. coupling of compounds (II) with 1-(2-ethoxyethyl)-2-(piperidin-4-yl)-1H-benzo[d]imidazole (III) in presence of a suitable base to form compound (IV); 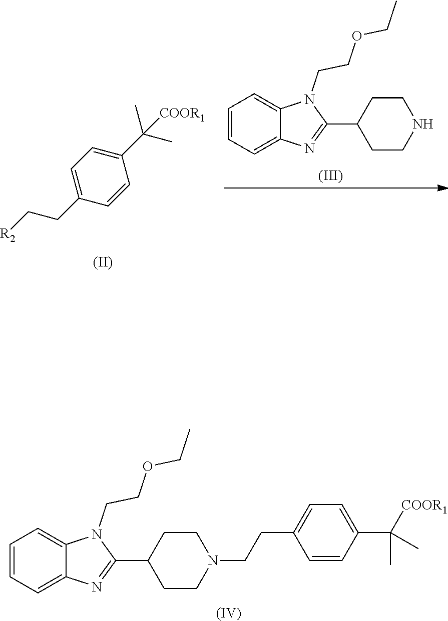
wherein, R₁ is C₁-C₄ alkyl; R₂ is halo or SO₃CH₃
and
vi. hydrolysis of compound (IV) with a suitable base to obtain Bilastine (I), wherein the Bilastine (1) obtained has total impurities less than 0.5% (w/w).

2. The process as claimed in claim 1, wherein the suitable acetylating reagent of step i) is selected from the group consisting of isobutyryl chloride, isobutyl bromide, acetyl chloride, acetyl bromide and acetic anhydride.

3. The process as claimed in claim 1, wherein the suitable oxidative reagent of step ii) is selected from the group consisting of iodine (I₂), tert-butyl hydroperoxide (TBHP), phenyliodonium diacetate (PhI(OAc)₂), (2,2,6,6-tetramethylpiperidin-1-yl) oxidanyl (TEMPO), Bromine (Br₂), trimethyl orthoformate (TMOF), zinc (II) bromide (ZnBr₂), phenyliodonium diacetate (PhI(OAc)₂) in trimethyl orthoformate, iodine (I₂)/trimethyl orthoformate (TMOF)/sulfuric acid, iodine chloride and trimethyl orthoformate (TMOF).

4. The process as claimed in claim 1, wherein the suitable metal halide of step ii) is selected from the group consisting of iodine chloride, sodium chloride, sodium bromide, sodium iodide, potassium chloride, potassium bromide and potassium iodide.

5. The process as claimed in claim 1, wherein the suitable base is selected from the group consisting of hydroxides of alkali and alkaline metals such as lithium hydroxide, sodium hydroxide, potassium hydroxide and the like; carbonates of alkali and alkaline metals such as sodium carbonate, potassium carbonate and the like; and bicarbonates of alkali and alkaline metals such as sodium bicarbonate, potassium bicarbonate and the like; “alkali metal alkoxides” such as sodium methoxide, sodium ethoxide, sodium tert-butoxide, potassium methoxide, potassium ethoxide, potassium tert-butoxide and the like.

6. The process as claimed in claim 1, wherein the suitable reagent of step iv) is selected from the group consisting of chlorinating agent or sulfinyl reagent; wherein chlorinating agent is selected from phosphoryl chloride, thionyl chloride, oxalyl chloride, methane sulfonyl chloride, trichloromethane and sulfonyl chloride; sulfinyl reagent is selected from methane sulfonyl chloride, p-toluene sulfonylchloride, mesyl chloride, p-toluenesulfonyl bromide and methane sulfonyl bromide.

7. A process for the purification of Bilastine (I), comprising:
a) providing Bilastine (I), in a suitable solvent.
b) heating the reaction mixture to a suitable temperature.
c) cooling the reaction mass; and
d) isolating pure Bilastine (I).

8. The process as claimed in claim 7 wherein, the suitable solvent is selected from water, methanol, ethanol, propanol, isopropanol, n-butanol, dichloromethane, dichloroethane, chloroform, carbon tetrachloride, dimethylacetamide (DMA), dimethylformamide (DMF), dimethylsulfoxide (DMSO), N-methylpyrrolidone (NMP), n-hexane, n-heptane, cyclohexane, pet ether, toluene, pentane, cycloheptane, methyl cyclohexane and m-, o-, or p-xylene or mixtures thereof.

9. A compound of formula (VII) 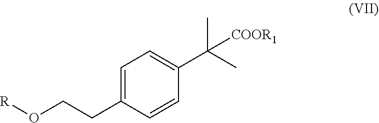
where R is defined as COCH(CH₃)₂, or COCH₃, R₁ is C₁-C₄ alkyl.

10. The compound as claimed in claim 9, which is selected from

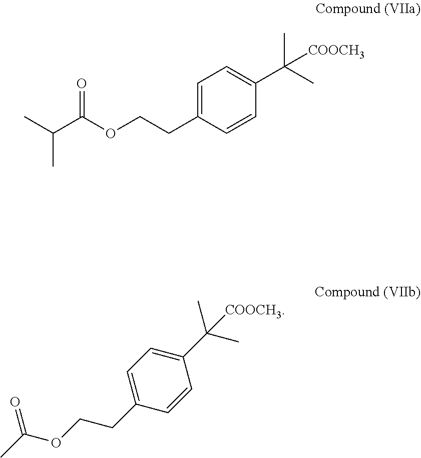 [SMILES: COC(=O)C(C)(C)c1ccc(CCOC(=O)C(C)C)cc1 ; COC(=O)C(C)(C)c1ccc(CCOC(C)=O)cc1]

11. The process as claimed in claim 1, wherein a suitable solvent is selected from water, methanol, ethanol, propanol, isopropanol, n-butanol, dichloromethane, dichloroethane, chloroform, carbon tetrachloride, dimethylacetamide (DMA), dimethylformamide (DMF), dimethylsulfoxide (DMSO), N-methylpyrrolidone (NMP), n-hexane, n-heptane, cyclohexane, pet ether, toluene, pentane, cycloheptane, methyl cyclohexane and m-, o-, or p-xylene or mixtures thereof.
